(19) United States
(12) Patent Application Publication
(10) Pub. No.: US 2008/0181879 A1
(43) Pub. Date: Jul. 31, 2008

(54) FIBRIN GEL FOR CONTROLLED RELEASE OF TGF-BETA AND USES THEREOF

(75) Inventors: Isabelle Catelas, Evanston, IL (US); Sam L. Helgerson, Lincolnshire, IL (US)

Correspondence Address: Baxter Healthcare Corporation;Law Department - 1D-15, One Baxter Way, Westlake Village, CA, 91362-3411, US

(73) Assignees: BAXTER INTERNATIONAL INC., Deerfield, IL (US); BAXTER HEALTHCARE S.A., Wallisellen (CH)

(21) Appl. No.: 12/016,986

(22) Filed: Jan. 18, 2008

Related U.S. Application Data
(60) Provisional application No. 60/881,452, filed on Jan. 18, 2007.
(60) Provisional application No. 60/934,457, filed on Jun. 13, 2007.

(51) Int. Cl. A61K 38/48; A61K 38/18; A61P 9/00; A61P 19/00

(52) U.S. Cl. 424/94.64; 514/12

(57) ABSTRACT

The present invention relates, in general, to fibrin sealants, which contain transforming growth factor-beta (TGF-β) for controlled release in situ for therapeutic applications, including musculoskeletal disorders, such as bone and cartilage disorders, soft tissue disorders and cardiovascular diseases.

This application claims the priority benefit of U.S. Provisional Patent application No. 60/881,452, filed Jan. 18, 2007 and Provisional Patent Application No. 60/934,457, filed Jun. 13, 2007, herein incorporated by reference in their entirety.

### FIELD OF THE INVENTION

The present invention relates, in general, to fibrin sealants, which contain transforming growth factor-beta (TGF-β) for controlled release in situ for therapeutic applications, including musculoskeletal and cardiovascular diseases.

### BACKGROUND OF THE INVENTION

Fibrin sealants are a type of surgical “glue” that is made from human blood-clotting proteins, and that is typically used during surgery to control bleeding. The ingredients in these sealants interact during application to form a stable clot composed of a blood protein fibrin. Fibrin sealants are presently used during surgery for several different purposes: to control bleeding in the area where the surgeon is operating, to speed wound healing, to seal off hollow body organs or cover holes made by standard sutures, to provide slow-release delivery of medications to tissues exposed during surgery.

Fibrin sealants generally consist of two human plasma-derived components: (a) a highly concentrated Fibrinogen Complex (FC) composed primarily of fibrinogen and fibronectin along with catalytic amounts of Factor XIII and plasminogen and (b) a high potency thrombin. Fibrin sealants may also contain aprotinin. By the action of thrombin, (soluble) fibrinogen at first is converted into fibrin monomers which aggregate spontaneously and form a so-called fibrin clot. Simultaneously, factor XIII (FXIII) present in the solution is activated by thrombin in the presence of calcium ions to factor XIIIa. The aggregated fibrin monomers and any remaining fibronectin possibly present are cross-linked to form a high polymer by new peptide bonds forming. By this cross-linking reaction, the strength of the clot formed is substantially increased. Generally, the clot adheres well to wound and tissue surfaces, which leads to the adhesive and haemostatic effect. (U.S. Pat. No. 7,241,603). Therefore, fibrin adhesives are frequently used as two-component adhesives which comprise a fibrinogen complex (FC) component together with a thrombin component which additionally contains calcium ions.

A particular advantage of a fibrin sealant is that the adhesive/gel does not remain at its site of application as a foreign body, but is completely resorbed just as in natural wound healing, and is replaced by newly formed tissue. Various cells, e.g., macrophages and, subsequently, fibroblasts migrate into the gel, lyse and resorb the gel material and form new tissue. Fibrin sealants have been used to form fibrin gels in situ, and these fibrin gels have been used for delivery of cells and growth factors (Cox et al., Tissue Eng 10(5-6): 942-954, 2004; Wong et al., Thromb Haemost 89: 573-582, 2003).

For tissue repair, it is desirable to localize growth factors and cells in a matrix such as a fibrin gel. For example, fibrin matrix has been used for delivery of TGF-β in various complex mixtures including fetal bovine serum, coral granules, and liposomes (Fortier et al., Am J Vet Res 58(1): 66-70, 1997; Arnaud et al., Chirurgie Plastique Esthetique 39(4): 491-498, 1994; Arnaud et al. Calcif Tissue Int 54: 493-498, 1994; Giannoni et al., Biotechnology and Bioengineering 83(1): 121-123, 2003). Alternative means to deliver growth factors from fibrin gels involve conjugates comprising transglutaminase substrates, antibodies, and VEGF fragments bound to the growth factors (See, for example U.S. Pat. No. 6,506,365; U.S. Pat. No. 6,713,453 and US Patent Publication 2003/0012818, incorporated herein by reference in their entirety). Additionally, fibrin gels have been shown to induce cell growth (e.g., human mesenchymal stem cell (HMSC)) and proliferation as well as, to some extent, osteogenic differentiation, depending on the concentrations of FC and thrombin in the matrix (Catelas et al., Tissue Eng 12(8): 2385-2396, 2006).

The ability of fibrin sealants to deliver growth factors to a particular site in the body is beneficial, but proper regrowth of tissue often requires a continuous/steady supply of growth factor or cytokine delivered at a specific rate to the site so that proper treatment is ensured. This is especially true if the therapeutic protein has a short half-life in vivo. Fibrin sealants currently in use provide for some delayed release of the seeded drug or agent, but the ability to extend the life of the agent in the sealant would improve the long-term tissue repair in vivo.

Thus, there remains a need in the art to develop an effective means to deliver growth factor in vivo for treatment of various conditions and disorders, to develop improved methods for controlled release of growth factors from a fibrin gel.

### SUMMARY OF THE INVENTION

The present invention provides compositions of fibrin sealant comprising a transforming growth factor-beta (TGF-β) for controlled release of the growth factor in vitro and in vivo. The invention also provides a method to modify the release of TGF-β protein from a fibrin sealant by modifying the content of fibrinogen complex component used to formulate the sealant. For the treatment of a condition or disorder, it is contemplated that the TGF-β, once released from the fibrin sealant, retains its biological activity such that the TGF-β can mediate its expected biological activity in vitro or in vivo.

In one aspect, the invention provides a method for modifying the release of a transforming growth factor-beta (TGF-β) protein, said protein selected from the group consisting of TGF-β1, TGF-β2 and TGF-β3, from a fibrin sealant, wherein the fibrin sealant is produced by admixture of a fibrinogen complex component, a thrombin component and a TGF-β component, the method comprising, a) determining the amount of TGF-β released from a first fibrin sealant having a known initial amount of TGF-β and a known final concentration of fibrinogen complex, and b) modifying the known final concentration of fibrinogen complex used in the first fibrin sealant of step (a) to produce a second fibrin sealant, wherein increasing the concentration of the fibrinogen complex in the second sealant compared to the known final concentration of fibrinogen complex in the first sealant decreases the rate of TGF-β release from the second sealant as compared to the release of TGF-β from the first sealant of step (a), and wherein the second sealant has the same initial amount of TGF-β as the first sealant in step (a).

In a related aspect, the invention provides a method for modifying the release of a TGF-β protein, said protein selected from the group consisting of TGF-β1, TGF-β2 and TGF-β3, from a fibrin sealant, wherein the fibrin sealant is produced by admixture of a fibrinogen complex component, a thrombin component and a TGF-β3 component, the method comprising, a) determining the amount of TGF-β3 released from a first fibrin sealant having a known initial amount of TGF-β and a known final concentration of fibrinogen complex, and b) modifying the known final concentration of fibrinogen complex used in the first fibrin sealant in of step (a) to produce a second fibrin sealant, wherein decreasing the concentration of fibrinogen complex in the second sealant compared to the known final concentration of fibrinogen complex in the first sealant increases the rate of TGF-β release from the second sealant as compared to the release of TGF-β from the first sealant of step (a), and wherein the second sealant has the same initial amount of TGF-β as the first sealant in step (a).

In one embodiment, the final fibrinogen complex concentration in the first or second sealant is within the range of about 1 mg/ml to about 150 mg/ml. In a related embodiment, the final fibrinogen complex concentration in the first or second sealant is within the range of about 5 mg/ml to about 75 mg/ml.

In another embodiment, it is contemplated that the final fibrinogen complex concentration in the first fibrin sealant differs from the final fibrinogen complex concentration in the second sealant by about 1 mg/ml to about 149 mg/ml. In a further embodiment, the final fibrinogen complex concentration in the first fibrin sealant differs from the fibrinogen complex concentration in the second sealant by about 5 mg/ml to about 75 mg/ml. In yet another embodiment, the final fibrinogen complex concentration in the first fibrin sealant differs from the fibrinogen complex concentration in the second sealant by about 10 mg/ml to about 60 mg/ml.

It is contemplated that in some embodiments, the final concentration of the thrombin component in the first or second sealant is within the range of about 1 IU/ml to 250 IU/ml. In another embodiment, the final concentration of TGF-β in the first or second sealant is in the range of about 1 ng/ml to about 1 mg/ml.

In another aspect, the invention contemplates a method for the controlled release of a TGF-β protein, said protein selected from the group consisting of TGF-β1, TGF-β2 and TGF-β3, in a patient in need thereof, comprising administering to said patient a fibrin sealant comprising TGF-β, wherein at least 25% of the TGF-β is retained in the fibrin sealant for at least 3 days.

In a related aspect, the invention provides a method for the controlled release of a TGF-β protein, said protein selected from the group consisting of TGF-β1, TGF-β2 and TGF-β3, in a patient in need thereof, comprising administering to said patient a fibrin sealant comprising TGF-β, wherein at least 20% of the TGF-β is retained in the fibrin sealant for at least 10 days.

It is contemplated that the TGF -β released from the fibrin sealant is biologically active.

In some embodiments, at least 35% to 90% of the TGF-β is retained for at least 3 days. In a related embodiment, at least 45% to 75% of the TGF-β is retained in the fibrin sealant for at least 3 days. In a further embodiment, at least 60% of the TGF-β is retained in the fibrin sealant for at least 3 days.

In another embodiment, at least 25% to 75% of the TGF-β is retained in the fibrin sealant for at least 10 days. In a related embodiment, at least 45% to 55% of said TGF-β is retained in the fibrin sealant for at least 10 days.

In a related embodiment, it is contemplated that the fibrin sealant may have release kinetics of the above ranges for either or both of 3 days or 10 days.

In one embodiment, the fibrin sealant is produced by combining a fibrinogen complex (FC) component and a thrombin component in admixture. In another embodiment, the TGF-β is added to the FC component before admixture of the FC component with the thrombin component. In a further embodiment, the TGF-β is added to the thrombin component. In a still further embodiment, TGF-β is added to the mixture of FC and thrombin before the components are allowed to form the fibrin gel.

In a related embodiment, it is contemplated that the TGF-β release may decrease by a regular amount each day. For example, the TGF-β amount in the fibrin sealant may decrease by about 1% a day, by about 2% a day, by about 3% a day, by about 4% a day, by about 5% a day, by about 6% a day, by about 7% a day, by about 8% a day, by about 9% a day or by about 10% a day, or the desired amount of release may be adjusted based on the fibrinogen complex concentration or thrombin concentration used to formulate the fibrin sealant.

The invention contemplates that the final concentration of the fibrinogen complex component in the sealant is within the range of about 1 mg/ml to about 150 mg/ml. It is also contemplated that, in some embodiments, the final concentration of the thrombin component in the sealant is within the range of about 1 IU/ml to 250 IU/ml. In one embodiment, the final fibrinogen complex concentration is about 5 mg/ml, about 10 mg ml, about 20 mg/ml or about 40 mg/ml, and the final thrombin concentration is about 2 IU/ml.

In one embodiment, the final TGF-β concentration in the sealant is from about 1 ng/ml to about 1 mg/ml.

In a further embodiment, it is contemplated that the TGF-β is TGF-β1. In one embodiment, when the TGF-β is TGF-β1, at least 60% of said TGF-β1 is retained in said fibrin sealant for at least 3 days, and wherein at least 25% of said TGF-β1 is retained in said fibrin sealant for at least 10 days.

In a related embodiment, it is contemplated that the TGF-β is TGF-β2. In one embodiment, when the TGF-β is TGF-β2, at least 25% of said TGF-β2 is retained in said fibrin sealant for at least 3 days.

In another embodiment, it is contemplated that the TGF-β is TGF-β3. In one embodiment, when the TGF-β is TGF-β3, at least 55% of said TGF-β3 is retained in said fibrin sealant for at least 3 days, and wherein at least 25% of said TGF-β3 is retained in said fibrin sealant for at least 10 days.

In a further aspect, the invention contemplates a method for treating a patient suffering from a disorder or disease which would benefit from in situ controlled release of a transforming growth factor beta (TGF-β) protein, said protein selected from the group consisting of TGF-β1, TGF-β2 and TGF-β3, said method comprising administering to said patient a fibrin sealant comprising the TGF-β protein, wherein the fibrin sealant provides a controlled release of the TGF-0 wherein at least 25% of the TGF-β is retained in the fibrin sealant for at least 3 days, and said TGF-β is released at a rate effective to treat said disorder or disease.

In a further aspect, the invention contemplates a method for treating a patient suffering from a disorder or disease which would benefit from in situ controlled release of a bioactive transforming growth factor beta (TGF-β) protein, said protein selected from the group consisting of TGF-β1, TGF-β2 and TGF-β3, said method comprising administering to said patient a fibrin sealant comprising the TGF-β protein, wherein the fibrin sealant provides a controlled release of the TGF-β wherein at least 20% of the TGF-0 is retained in the fibrin sealant for at least 10 days and said TGF-β is released at a rate effective to treat said disorder or disease.

The invention also provides for use of a fibrin sealant comprising a TGF-β protein, selected from the group consisting of TGF-β1, TGF-β2 and TGF-β3, in the manufacture of a medicament for treating a patient suffering from a disorder or disease which would benefit from in situ controlled release of a transforming growth factor beta (TGF-β) protein, wherein the fibrin sealant provides a controlled release of the TGF-β as above.

The invention contemplates that the release kinetics described above are applicable to the method for treating a patient who would benefit from in situ controlled release of a TGF-β protein, or to use of the fibrin sealant in the manufacture of a medicament to treat said patient.

In one aspect, the patient is suffering from a disease which would benefit from the controlled release of TGF-β in vivo which would be apparent to one of ordinary skill in the art. In one embodiment, the disease or disorder is selected from the group consisting of a musculoskeletal disease or disorder, a soft tissue disease or disorder and a cardiovascular disease. In one embodiment, the musculoskeletal disorder is a bone disease or a bone disorder. In a related embodiment, the musculoskeletal disorder is a cartilage disease or a cartilage disorder.

In one embodiment, the fibrin sealant is administered to a patient using methods well-known in the art, such as injection, spray, endoscopic administration or pre-formed gel, by itself or in combination with other materials, and other methods known to one of ordinary skill in the art.

The invention also provides a kit for preparing a fibrin sealant comprising a bioactive transforming growth factor beta (TGF-β) protein, said protein selected from the group consisting of TGF-β1, TGF-β2 and TGF-β3, and said fibrin sealant having a desired TGF-β release rate, the kit comprising, a) a first vial or first storage container containing a fibrinogen complex component, wherein the vial optionally comprises a TGF-β component, and b) a second vial or second storage container having a thrombin component, said kit optionally containing a third vial or third storage container having a TGF-β component when said first vial or first storage container does not include a TGF-β component, said kit further containing instructions for use thereof. The kit may also comprise instruments for use or administration of the fibrin sealant in vitro or in vivo.

Other features and advantages of the invention will become apparent from the following detailed description. It should be understood, however, that the detailed description and the specific examples, while indicating specific embodiments of the invention, are given by way of illustration only, because various changes and modifications within the spirit and scope of the invention will become apparent to those skilled in the art from this detailed description.

### BRIEF DESCRIPTION OF THE DRAWINGS

FIG. 1 shows the effects of TGF-β1 amount on its daily release from fibrin gels made of TISSEEL VH™ ([FC]=25 mg/ml, [Thrombin]=2 IU/ml)

FIG. 2 shows the effects of TGF-β1 amount on its cumulative release from fibrin gels made of TISSEEL VH™ ([FC]=25 mg/ml, [Thrombin]=2 IU/ml)

FIG. 3 shows the effects of FC concentration on TGF-β1 daily release from fibrin gels made of TISSEEL VH™ ([Thrombin]=2 IU/ml)

FIG. 4 shows the effects of FC concentration on TGF-β1 cumulative release from fibrin gels made of TISSEEL VH™ ([Thrombin]=2 IU/ml).

FIG. 5 shows the effects of FC concentration on TGF-β1 daily release from fibrin gels made of TISSEEL VH S/D™ ([Thrombin]=2 IU/ml).

FIG. 6 shows the effects of FC concentration on TGF-β1 cumulative release from fibrin gels made of TISSEEL VH S/D™ ([Thrombin]=2 IU/ml).

FIG. 7 shows the effects of Thrombin concentration on TGF-β1 daily release from fibrin gels made of TISSEEL VH™ ([FC]=25 mg/ml).

FIG. 8 shows the effects of Thrombin concentration on TGF-β1 cumulative release from fibrin gels made of TISSEEL VH™ ([FC]=25 mg/ml).

FIG. 9 shows the effects of TISSEEL VH™ lot number on TGF-β1 cumulative release from fibrin gels ([FC]=20 mg/ml, [Thrombin]=2 IU/ml).

FIG. 10 shows the effects of TISSEEL VH S/D™ lot number on TGF-β1 cumulative release from fibrin gels ([FC]=20 mg/ml, [Thrombin]=2 IU/ml).

FIG. 11 (biological activity of released TGF-β1) shows the proliferation of Human Mesenchymal Stem Cells (HMSC) cultured in monolayers with medium supernatant from TISSEEL VH™ fibrin gels with or without added TGF-β1 at day 3 or with freshly prepared medium with an additional 2 ng (1 ng/ml) of TGF-β1 (positive control). Results were normalized in Day 1.

FIG. 12 (biological activity of released TGF-β1) shows Alkaline Phosphatase (ALP) activity in HMSC cultured in medium supernatants from TISSEEL VH™ fibrin gels with added TGF-β1 (i.e. in medium containing released TGF-β1), compared to ALP activity in HMSC cultured in medium supernatants from fibrin gels with no added TGF-β1, and to ALP activity in HMSC cultured in medium that contained freshly added TGF-β1 (positive control). Results (first calculated in IU/ml) were normalized on proliferation.

### DETAILED DESCRIPTION OF THE INVENTION

The invention provides a fibrin gel containing TGF-β for controlled release in situ in therapeutic applications, including treatment of musculoskeletal diseases, such as bone and cartilage disorders, soft tissue disorders, and cardiovascular diseases. The invention contemplates that the TGF-β released from the gel retains its biological activity such that release from the fibrin sealant in vivo or in vitro modulates the desired biological activity. The invention also provides a method for determining the concentration of the FC component or thrombin component useful in formulating the fibrin sealant to obtain desired TGF-β release kinetics.

Unless otherwise defined, all technical and scientific terms used herein have the same meaning as commonly understood by one of ordinary skill in the art to which this invention belongs. The following references provide one of skill with a general definition of many of the terms used in this invention: Singleton, et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger, et al. (eds.), Springer Verlag (1991); and Hale and Marham, THE HARPER COLLINS DICTIONARY OF BIOLOGY (1991).

Each publication, patent application, patent, and other reference cited herein is incorporated by reference in its entirety to the extent that it is not inconsistent with the present disclosure.

It is noted here that as used in this specification and the appended claims, the singular forms “a,” “an,” and “the” include plural reference unless the context clearly dictates otherwise.

As used herein, the following terms have the meanings ascribed to them unless specified otherwise.

As used herein the terms “fibrin sealant,” “fibrin gel,” “fibrin adhesive,” “fibrin clot” or “fibrin matrix” are used interchangeably and refer to a three-dimensional network comprising at least a fibrinogen complex (FC) component and a thrombin component, which can act as a scaffold for cell growth and release of a bioactive materials over time.

As used herein the terms “controlled release” and “delayed release” have the same meaning and refer to retention of an agent (e.g., growth factor) in a fibrin gel. Controlled release is due not only to slow and steady secretion/release of the growth factor by diffusion or by dissociation of the bound growth factor and its subsequent diffusion from the gel, but is also due to the disintegration and enzymatic cleavage of the matrix.

As used herein, “in situ formation” refers to either formation at a physiological temperature and at the site of injection in the body or to formation of the fibrin sealant at appropriate in vitro conditions. This term is typically used to describe the formation of covalent linkages between precursor molecules in the fibrin sealant, which are substantially not crosslinked prior to and at the time of administration.

As used herein, “fibrinogen complex component” refers to the fibrin/fibrinogen solution which is mixed with thrombin resulting in a clot-like fibrin sealant. The fibrinogen complex (FC) is composed mainly of fibrinogen and fibronectin, and may also contain catalytic amounts of FXIII and plasminogen. The fibrinogen complex component may also be referred to as Sealer Protein.

As used herein, “thrombin component” refers to the thrombin solution which is mixed with fibrinogen complex component which results in a clot-like fibrin sealant.

As used herein, “transforming growth factor-beta component” or “TGF-β component” refers to the addition of the growth factor in solution to the liquid form of the fibrin sealant. Each of the TGF-β component, the FC complex component and the thrombin component may be added separately to form the fibrin sealant comprising TGF-β. Optionally, the TGF-β component is added to the liquid FC complex component before admixture with the thrombin component.

As used herein, “recombinant human TGF-β” refers to recombinant human transforming growth factor-β (rh TGF-β) obtained via recombinant DNA technology. It may be produced by any method known in the art.

As used herein the term “bioactive” or “biologically active” refers to the biological property wherein a protein, e.g., a TGF-β protein, in a solution or in a fibrin sealant exhibits the same or similar biological activity when compared to a naturally expressed (i.e., when expressed either recombinantly or in vivo) protein.

As used herein a “detectable moiety,” “detectable label” or “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavidin, dioxigenin, haptens and proteins for which anti-sera or monoclonal antibodies are available, or nucleic acid molecules with a sequence complementary to a target. The detectable moiety often generates a measurable signal, such as a radioactive, chromogenic, or fluorescent signal, that can be used to quantitate the amount of bound detectable moiety in a sample.

### Fibrin Sealants

Many forms of fibrin are available for use as a fibrin sealant. Fibrin gels can be synthesized from autologous plasma, cryoprecipitated plasma (e.g. fibrin glue kits, which are available commercially), fibrinogen purified from plasma, and recombinant fibrinogen and factor XIIIa. Each of these materials provides a fundamentally similar matrix, with small variations in the biochemical compositions. Sierra D H, J Biomater Appl, 7, 309-352 (1993). Similarities between these materials exist both in specific enzymatic bioactivity and general healing responses.

The fibrin gel useful in the invention is formed from a fibrin sealant, which consists of two main components: fibrinogen complex (FC) and thrombin. The FC is composed mainly of fibrinogen and fibronectin, and may also contain catalytic amounts of FXIII and plasminogen. The FC and thrombin components are generally derived from human plasma, but may also be produced by recombinant/genetic engineering techniques. Examples of Fibrin Sealants are described in U.S. Pat. No. 5,716,645; U.S. Pat. No. 5,962,405; U.S. Pat. No. 6,579,537 and include TISSEEL VH™ and TISSEEL VH S/D™ (Baxter Healthcare, Deerfield, Ill.).

To form the fibrin gel, the FC is first reconstituted, thawed or otherwise prepared according to package instructions, further diluted as needed using dilution buffers and therapeutic agent is added to the liquid FC. Most commercially available fibrin sealants include an inhibitor of gel lysis such as aprotinin, which can be added to the FC at the discretion of the user. A description of aprotinin and other gel lysis inhibitors is provided in WO 99/11301. The thrombin component is also reconstituted to liquid form using CaCl₂ solution, further diluted as needed using dilution buffers. It is contemplated that the thrombin component is mixed with the FC component further comprising a TGF-β to form the fibrin gel. Fibrin sealants have also been designed which lack the aprotinin ingredient (EVICEL™, Ethicon, Inc, New Jersey).

Additional methods for producing fibrinogen-containing preparations that can be used as tissue adhesives include, production from cryoprecipitate, optionally with further washing and precipitation steps with ethanol, ammonium sulphate, polyethylene glycol, glycine or beta-alanine, and production from plasma within the scope of the known plasma fractionation methods, respectively (cf., e.g., “Methods of plasma protein fractionation”, 1980, ed.: Curling, Academic Press, pp. 3-15, 33-36 and 57-74, or Blomb ck B. and M., “Purification of human and bovine fibrinogen”, Arkiv Kemi 10, 1959, p. 415 f.). Fibrin sealant may also be made using a patients own blood plasma. For example, the CRYOSEAL® (Thermogenesis Corp., Rancho Cordova, Calif.) or VIVOSTAT® (Vivolution A/S, Denmark) fibrin sealant systems enables the production of autologous fibrin sealant components from a patient's blood plasma. The components of Fibrin Sealants are available in lyophilized, deep-frozen liquid, or liquid form.

The components of the fibrin gel are added at appropriate concentrations to provide the type of controlled release desired. The FC component may be added in varying concentrations, including but not limited to 5 mg/ml, 10 mg/ml, 15 mg/ml, 20 mg/ml, 25 mg/ml, 30 mg/ml, 35 mg/ml, 40 mg/ml, 45 mg/ml, 50 mg/ml, up to 150 mg/ml (final concentrations in the gels), or in intermediate concentrations as necessary. Further, the concentration of FC component may be combined with any appropriate concentration of thrombin component, including, but not limited to 1 IU/ml, 2 IU/ml, 5 IU/ml, 7 IU/ml, 10 IU/ml, 15 IU/ml, 20 IU/ml, 25 IU/ml, 30 IU/ml, 35 IU/ml, 40 IU/ml, 50 IU/ml, 60 IU/ml, 70 IU/ml, 80 IU/ml, 90 IU/ml, 100 IU/ml, 120 IU/ml, 140 IU/ml, 150 IU/ml, 175 IU/ml, 200 IU/ml, 225 IU/ml and 250 IU/ml.

It is contemplated that a second agent such as TGF-β is added to the fibrin sealant composition in order to make a controlled release system for the therapeutic agent. The TGF-βt may be added in any concentration that provides an adequate delayed release formulation, within a range of 1 ng/ml to 1 mg/mL of TGF-β. Exemplary concentrations of TGF-β in the fibrin sealant include, but are not limited to 1 ng/ml, 5 ng/ml, 10 ng/ml, 15 ng/ml, 20 ng/ml, 40 ng/ml, 50 ng ml, 100 ng/ml, 250 ng/ml, 500 ng/ml, 1 μg/ml, 5 μg/ml, 10 μg/ml, 25 μg/ml, 50 μg/ml, 100 μg/ml, 250 μg/ml, 500 μg/ml, 750 μg/ml and 1 mg/ml.

It is contemplated that the concentration of FC or thrombin used in the fibrin sealant are such that the TGF-β added in the fibrin gel is released in a therapeutically effective amount over the course of several days to weeks. In one aspect, the TGF-β is released from the fibrin gel for 1 day, 2 days, 3 days, 4 days, 5 days, 6 days, 7 days, 8 days, 9 days, 10 days, 11 days, 12 days, 13 days, 14 days, 15 days, 16 days, 17 days 18 days, 19 days, 20 days, or longer.

The TGF-β is released form the fibrin sealant in a controlled or delayed release manner, such that the TGF-β is available in situ over a sustained period of time. It is contemplated that the TGF-β release may decrease by a regular amount each day, for example, the TGF-0 levels may decrease by about 1% a day, by about 2% a day, by about 3% a day, by about 4% a day, by about 5% a day, by about 6% a day, by about 7% a day, by about 8% a day, by about 9% a day or by about 10% a day or more. In a related embodiment, it is contemplated that at least 25% of TGF-β is retained in the fibrin gel for at least 3 days. In a further embodiment, at least 35% to 90%, at least 45% to 75%, or at least 60% of the TGF-β is retained in the fibrin gel for at least 3 days. It is further contemplated that at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the TGF-β is retained in the fibrin gel for at least 3 days.

In another embodiment, at least 20% of the TGF-β is retained in the fibrin gel for at least 10 days. In a further embodiment, at least 25% to 75% or 45% to 55% of the TGF-β is retained for at least 10 days. It is further contemplated that at least 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74% or 75% of the TGF-β is retained in the fibrin gel for at least 10 days.

The invention provides a method to formulate a fibrin sealant having desired release kinetics by modifying the concentration of the components of the fibrin sealants. In one aspect, the method contemplates determining the amount of TGF-β released from a first fibrin sealant having a known initial amount of TGF-β and a known final concentration of fibrinogen complex, modifying the known final concentration of fibrinogen complex used in the first fibrin sealant in of step (a) to produce a second fibrin sealant, wherein increasing or decreasing the concentration of fibrinogen complex in the second sealant compared to the known final concentration of fibrinogen complex in the first sealant adjusts the rate of TGF-β release from the second sealant as compared to the release of TGF-β from the first sealant of step, and wherein the second sealant has the same initial amount of TGF-β as the first sealant in step.

In one embodiment, the final fibrinogen complex concentration in the first or second sealant is within the range of about 1 mg/ml to about 150 mg/ml. The method of claim 1 or 2 wherein the final fibrinogen complex concentration in the first or second sealant is as set out above. In a related embodiment, the FC concentration of the first fibrin sealant differs from the final FC concentration in the second sealant by about 1 mg/ml to about 149 mg/ml, by about 5 mg/ml to about 75 mg/ml, or by about 10 mg/ml to about 60 mg/ml. In a further embodiment, the FC concentration FC concentration of the first fibrin sealant differs from the final FC concentration in the second sealant by about 2 mg/ml, 3 mg/ml, 4 mg/ml, 5 mg/ml, 10 mg/ml, 15 mg/ml, 20 mg/ml, 25 mg/ml, 30 mg/ml, 35 mg/ml 40 mg/ml, 45 mg/ml, 50 mg/ml, or any amount between these concentrations, up to about 149 mg/ml.

It is contemplated that the fibrin sealant useful in the invention may be combined with additional materials or agents for purposes of in vitro or in vivo use. Such agents include additional therapeutic agents, including, but not limited to, growth factors, cytokines, chemokines, a blood clotting factor, an enzyme, a chemokine, a soluble cell-surface receptor, a cell adhesion molecule, an antibody, a hormone, a cytoskeletal protein, a matrix protein, a chaperone protein, a structural protein, a metabolic protein, and others known in the art. See, for example, Physicians Desk Reference, 62^{nd} Edition, 2008, Thomson Healthcare, Montvale, N.J.

Additional materials useful in the fibrin sealant include materials that could be combined with the sealant for bone or cartilage disease which may be load bearing materials, including but not limited to, polymers, coral, ceramics, glass, metals, bone-derived materials, hydroxyapatite, synthetic scaffolds materials, combinations of these materials, and other materials known in the art. See, e.g., Guehennec et al., (European Cells and Materials, 8:1-11, 2004), U.S. Pat. No. 7,122,057 and U.S. Pat. No. 6,696,073.

In one embodiment, the fibrin gels can be used as a carrier system to deliver biologically active TGF-β after reversed-binding and in a controlled manner by adjusting the concentrations of FC and thrombin.

In one embodiment of the invention, when the fibrin gels are made of TISSEEL Vapor Heated (TISSEEL VH™) using FC at 25 mg/ml and Thrombin at 2 IU/ml (final concentrations in the gels), at least about 80% of the added TGF-β1 in retained in the gels after 3 days, and at least about 48% of the added TGF-β1 is retained in the gels after 10 days. TGF-β release amount is proportional to the amount of growth factor added in the gels.

In another embodiment of the invention, when the fibrin gels are made of TISSEEL VH™ using FC at 5 mg/ml and Thrombin at 2 IU/ml (final concentrations in the gels), at least about 60% of the added TGF-β1 is retained in the gels after 3 days, and at least about 25% of the added TGF-β1 is retained in the gels after 10 days. In another embodiment of the invention, when the fibrin gels are made of TISSEEL Vapor Heated Solvent/Detergent (TISSEEL VH S/D™) using FC at 5 mg/ml and Thrombin at 2 IU/ml (final concentrations in the gels), at least about 35% of the added TGF-β1 is retained in the gels after 3 days, and less than 7% is retained in the gels after 10 days (i.e. almost completely released). Therefore, the retention increases with higher FC concentrations when using fibrin gels made either of TISSEEL VH™ or TISSEEL VH S/D™.

In another embodiment of the invention, TGF-β1 release from fibrin gels made of TISSEEL VH™ with 2, 10 and 50 IU/ml of Thrombin (with 25 mg/ml of FC, final concentrations in the gels) is similar, suggesting that Thrombin concentration has a lesser effect than FC concentration on TGF-β1 release. TGF-β1 release is only significantly higher with the highest Thrombin concentration (250 IU/ml, final concentration in the gels), suggesting an effect of the gel structure, with a more heterogenous structure.

In another embodiment of the invention, when the fibrin gels are made of TISSEEL VH™ of different lot numbers, using FC at 20 mg/ml and Thrombin at 2 IU/ml (final concentrations in the gels), at least 67% of TGF-β1 is retained in the gels from one lot after 10 days, 39% from another lot and none from a third lot. One difference between these lots is the Factor XIII content (42.2 U/ml, 33.9 U/ml and <1 U/ml, respectively). In another embodiment of the invention, when the fibrin gels are made of TISSEEL VH S/D™ of different lot numbers, using FC at 20 mg/ml and Thrombin at 2 IU/ml (final concentrations in the gels), at least 20% of TGF-β1 is retained in the gels from one lot after 10 days, and none from the other two lots. The Factor XIII content of these lots were all lower than 3 U/ml.

In another embodiment of the invention when TGF-β2 is added to fibrin gels made of TISSEEL VH™ using FC at 5 mg/ml and Thrombin at 2 IU/ml (final concentrations in the gels), at least about 25% of the added TGF-β2 is retained in the gels after 3 days. Retention increases with FC concentration. When TGF-β3 is added to fibrin gels made of TISSEEL VH™ using FC at 5 mg/ml and Thrombin at 2 IU/ml (final concentrations in the gels), at least 55% of the added TGF-β3 is retained in the gels after 3 days and 25% after 10 days.

It will be understood to one of ordinary skill in the art that the embodiments set out above are exemplary embodiments of TGF-β release from a commercially available fibrin sealant and are not meant to limit the invention in any way.

### TGF-β Protein

TGF-beta exists in at least five isoforms, known as TGF-β1, TGF-β2, TGF-β3, TGF-β4, TGF-β5. Their amino acid sequences display homologies on the order of 70-80%.TGF-beta-1 is the prevalent form and is found almost ubiquitously while the other isoforms are expressed in a more limited spectrum of cells and tissues. TGF-β1, TGF-β2 and TGF-β3 appear to have distinct functions in bone morphogenesis (Fagenholz et al., J Craniofacial Surg. 12:183-190, 2001). The 3-dimensional structure of TGF-β1 is described in Hinck et al., Biochemistry 35: 8517-8534, 1996. The 3-dimensional structure of TGF-β2 is described in Daopin et al., Science 257: 369-373, 1992. The 3-dimensional structure of TGF-03 is described in Mittl et al., Protein Sci 5: 1261-1271, 1996.

In another embodiment of the invention, the biological activity of the released TGF-β1 from fibrin gels was tested. The change of Human Mesenchymal Stem Cell (HMSC) morphology into a more squared to polygonal shape after culture in monolayers in medium supernatants from gels with added TGF-β1 (i.e., in medium containing released TGF-β1) indicates cell differentiation, in parallel to a tendency to exhibit lower proliferation compared to cells cultured in medium supernatants from gels with no added TGF-β1. Alcian blue positive staining of the cells cultured in medium supernatants from gels with added TGF-β1 (i.e. in medium containing released TGF-β1) indicates that HMSC start to undergo chondrogenesis. Markers of early and late osteogenic differentiation, i.e. Alkaline Phosphatase (ALP) activity and Alizarin Red staining, respectively, remain negative. These changes in cell morphology, proliferation and chondrogenic differentiation demonstrate that TGF-131 is still biologically active after its release from the gels.

The TGF-β molecules useful for the present invention include the full-length protein, precursors of the protein, subunits or fragments of the protein, and functional derivatives thereof. Reference to TGF-β is meant to include all potential forms of such proteins, including naturally-derived protein preparations.

According to the present invention, the term recombinant TGF-β does not underlie a specific restriction and may include any TGF-β, heterologous or naturally occurring, obtained via recombinant DNA technology, or a biologically active derivative thereof. In certain embodiments, the term encompasses proteins and nucleic acids, e.g., gene, pre-mRNA, mRNA, and polypeptides, polymorphic variants, alleles, mutants, and interspecies homologs that: (1) have an amino acid sequence that has greater than about 60% amino acid sequence identity, 65%, 70%, 75%, 80%, 85%, 90%, 91%, 92%, 93%, 94%, 95%, 96%, 97%, 98% or 99% or greater amino acid sequence identity, over a region of at least about 25, 50, 100, 200, 300, 400, or more amino acids, to a TGF-β1, -β2 or -β3 polypeptide encoded by a referenced nucleic acid or an amino acid sequence described herein; (2) specifically bind to antibodies, e.g., polyclonal antibodies, raised against an immunogen comprising a referenced amino acid sequence as described herein immunogenic fragments thereof, and conservatively modified variants thereof; (3) specifically hybridize under stringent hybridization conditions to a nucleic acid encoding a referenced amino acid sequence as described herein, and conservatively modified variants thereof; (4) have a nucleic acid sequence that has greater than about 95%, greater than about 96%, 97%, 98%, 99%, or higher nucleotide sequence identity, over a region of at least about 25, 50, 100, 150, 200, 250, 500, 1000, or more nucleotides (up to the full length sequence of 1218 nucleotides of the mature protein), to a reference nucleic acid sequence as described herein.

A polynucleotide or polypeptide sequence is typically from a mammal including, but not limited to, primate, e.g., human; rodent, e.g., rat, mouse, hamster; cow, pig, horse, sheep, or any other mammal. The nucleic acids and proteins of the invention can be recombinant molecules (e.g., heterologous and encoding the wild type sequence or a variant thereof, or non-naturally occurring). For the structure of human TGF-β refer to Genbank Database maintained by the National Center for Biotechnology Information (NCBI): Human TGF β-1, Genbank Accession No. NP_{—}000651, Human TGF β-2, Genbank Accession No. NP_{—}003229, TGF β-3 Genbank Accession No. NP_{—}003230.

The production of TGF-β may include any method known in the art for (i) the production of recombinant DNA by genetic engineering, e.g. via reverse transcription of RNA and/or amplification of DNA, (ii) introducing recombinant DNA into prokaryotic or eukaryotic cells by transfection, e.g. via electroporation or microinjection, (iii) cultivating said transformed cells, e.g. in a continuous or batchwise manner, (iv) expressing TGF-β, e.g. constitutively or upon induction, and (v) isolating said TGF-β, e.g. from the culture medium or by harvesting the transformed cells, in order to obtain purified TGF-β, e.g. via anion exchange chromatography or affinity chromatography.

The TGF-β can be produced by expression in a suitable prokaryotic or eukaryotic host system characterized by producing a pharmacologically acceptable TGF-β molecule. Commonly used host cells include: Prokaryotic cells such as gram negative or gram positive bacteria, i.e., any strain of E. coli, Bacillus, Streptomyces, Saccharomyces, Salmonella, and the like. Examples of eukaryotic cells are insect cells such as D. Me1-2, Sf4, Sf5, Sf9, and Sf21 and High 5; plant cells and various yeast cells such as Saccharomyces and Pichia; mammalian cells, such as CHO (Chinese hamster ovary) cells; baby hamster kidney (BHK) cells; human kidney 293 cells; COS-7 cells, HEK 293, SK-Hep, and HepG2, and others known in the art. There is no limitation to the reagents or conditions used for producing or isolating TGF-β according to the present invention, and any system known in the art or commercially available can be employed. In a preferred embodiment of the present invention, TGF-β is obtained by methods as described in the state of the art.

A wide variety of vectors can be used for the preparation of the TGF-β and can be selected from eukaryotic and prokaryotic expression vectors well-known in the art. Examples of vectors for prokaryotic expression include, but are not limited to, plasmids such as pRSET, pET, pBAD, etc., wherein the promoters used in prokaryotic expression vectors include lac, trc, trp, recA, araBAD, etc. Examples of vectors for eukaryotic expression include, butr are not limited to: (i) for expression in yeast, vectors such as pAO, pPIC, pYES, pMET, using promoters such as AOX1, GAP, GAL1, AUG1, etc; (ii) for expression in insect cells, vectors such as pMT, pAc5, pIB, pMIB, pBAC, etc., using promoters such as PH, p10, MT, Ac5, OpIE2, gp64, polh, etc., and (iii) for expression in mammalian cells, vectors such as pSVL, pCMV, pRc/RSV, pcDNA3, pBPV, etc., and vectors derived from viral systems such as vaccinia virus, adeno-associated viruses, herpes viruses, retroviruses, etc., using promoters such as CMV, SV40, EF-1, UbC, RSV, ADV, BPV, and β-actin.

Host cells containing the polypeptide-encoding DNA or RNA are cultured under conditions appropriate for growth of the cells and expression of the DNA or RNA. Those cells which express the polypeptide can be identified, using known methods, and the recombinant protein isolated and purified, using known methods; either with or without amplification of polypeptide production. Identification can be carried out, for example, through screening genetically modified mammalian cells displaying a phenotype indicative of the presence of DNA or RNA encoding the protein, such as PCR screening, screening by Southern blot analysis, or screening for the expression of the protein. Selection of cells having incorporated protein-encoding DNA may be accomplished by including a selectable marker in the DNA construct and culturing transfected or infected cells containing a selectable marker gene under conditions appropriate for survival of only those cells that express the selectable marker gene. Further amplification of the introduced DNA construct can be affected by culturing genetically modified cells under conditions appropriate for amplification (e.g., culturing genetically modified cells containing an amplifiable marker gene in the presence of a concentration of a drug at which only cells containing multiple copies of the amplifiable marker gene can survive).

### Methods of Determining Protein Concentration in a Sample

Therapeutic proteins are often difficult to detect in serum samples due to their similarity to the endogenously produced, naturally-occurring protein. However, it is often beneficial to determine the amount of a therapeutic polypeptide, fragment, variant or analog thereof that has been administered to assess whether the therapeutic protein exhibits desired characteristics such as greater solubility or stability, resistance to enzyme digestion, improved biological half-life, and other features known to those skilled in the art. The method also allows for detection of authorized uses of therapeutic proteins which may be protected by intellectual property rights.

The present invention provides for a method to detect the release of TGF-β from a fibrin gel containing TGF-β and determine the release kinetics of the protein. The comparison of these release kinetics from fibrin sealants made using varying concentrations of fibrinogen complex component helps to determine the desired release rate for the therapeutic purpose. The ability to identify the amount of protein released from the fibrin sealant over time aids in determination of the optimal therapeutic based on half-life, absorption, stability, etc. The detection assay may be an enzyme linked immunosorbant assay (ELISA), a radioimmunoassay (RIA), a scintillation proximity assay (SPA), surface plasma resonance (SPR), or other binding assays known in the art.

Generally, for detecting the presence of TGF-β in a sample, the TGF-β is bound to a TGF-β binding agent, such as an antibody, soluble receptor or other protein or agent which binds TGF-β.

For the detection step, the TGF-β protein may be linked to a detectable moiety or a detectable label. Detectable moiety or label refers to a composition detectable by spectroscopic, photochemical, biochemical, immunochemical, or chemical means. The detectable moiety often generates a measurable signal, such as a radioactive, chromogenic, or fluorescent signal, that can be used to quantitate the amount of bound detectable moiety in a sample. The detectable moiety can be incorporated in or attached to the protein either covalently, or through ionic, van der Waals or hydrogen bonds, e.g., incorporation of radioactive nucleotides, or biotinylated nucleotides that are recognized by streptavidin. The detectable moiety may be directly or indirectly detectable. Indirect detection can involve the binding of a second directly or indirectly detectable moiety to the detectable moiety. For example, the detectable moiety can be the ligand of a binding partner, such as biotin, which is a binding partner for streptavidin. The binding partner may itself be directly detectable, for example, an antibody may be labeled with a fluorescent molecule. Selection of a method quantitation of the signal is achieved by, e.g., scintillation counting, densitometry, or flow cytometry.

Examples of labels suitable for use in the assay methods of the invention include, radioactive labels, fluorophores, electron-dense reagents, enzymes (e.g., as commonly used in an ELISA), biotin, digoxigenin, or haptens as well as proteins which can be made detectable, e.g., by incorporating a radiolabel into the hapten or peptide, or used to detect antibodies specifically reactive with the hapten or peptide. Also contemplated are proteins for which antisera or monoclonal antibodies are available, or nucleic acid molecules with a sequence complementary to a target, a nanotag, a molecular mass bead, a magnetic agent, a nano- or micro-bead containing a fluorescent dye, a quantum dot, a quantum bead, a fluorescent protein, dendrimers with a fluorescent label, a micro-transponder, an electron donor molecule or molecular structure, or a light reflecting particle.

Additional labels contemplated for use with present invention include, but are not limited to, fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold, colored glass or plastic beads (e.g., polystyrene, polypropylene, latex, etc.), and luminescent or chemiluminescent labels (e.g., Europium (Eu), MSD Sulfo-Tag).

The label may be coupled directly or indirectly to the desired component of the assay according to methods well known in the art. In a specific embodiment, the label is covalently bound to the component using an isocyanate or N-hydroxysuccinimide ester reagent for conjugation of an active agent according to the invention. In one aspect of the invention, bifunctional isocyanate reagents are used to conjugate a label to a biopolymer to form a label biopolymer conjugate without an active agent attached thereto. The label biopolymer conjugate may be used as an intermediate for the synthesis of a labeled conjugate according to the invention or may be used to detect the biopolymer conjugate. As indicated above, a wide variety of labels can be used, with the choice of label depending on sensitivity required, ease of conjugation with the desired component of the assay, stability requirements, available instrumentation, and disposal provisions. Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound.

The compounds useful in the method of the invention can also be conjugated directly to signal-generating compounds, e.g., by conjugation with an enzyme or fluorophore. Enzymes suitable for use as labels include, but are not limited to, hydrolases, particularly phosphatases, esterases and glycosidases, or oxidotases, particularly peroxidases. Fluorescent compounds suitable for use as labels include, but are not limited to, those listed above as well as fluorescein derivatives, rhodamine and its derivatives, dansyl, umbelliferone, eosin, TRITC-amine, quinine, fluorescein W, acridine yellow, lissamine rhodamine, B sulfonyl chloride erythroscein, ruthenium (tris, bipyridinium), europium, Texas Red, nicotinamide adenine dinucleotide, flavin adenine dinucleotide, etc. Chemiluminescent compounds suitable for use as labels include, but are not limited to, MSD Sulfa-TAG, Europium (Eu), Samarium (Sm), luciferin and 2,3-dihydrophthalazinediones, e.g., luminol. For a review of various labeling or signal producing systems that can be used in the methods of the present invention, see U.S. Pat. No. 4,391,904.

Means for detecting labels are well known to those of skill in the art and are dictated by the type of label to be detected. Thus, for example, where the label is radioactive, means for detection include a scintillation counter (e.g., radioimmunoassay, scintillation proximity assay) (Pitas et al., Drug Metab Dispos. 34:906-12, 2006) or photographic film, as in autoradiography. Where the label is a fluorescent label, it may be detected by exciting the fluorochrome with the appropriate wavelength of light and detecting the resulting fluorescence (e.g., ELISA, flow cytometry, or other methods known in the art). The fluorescence may be detected visually, by the use of electronic detectors such as charge coupled devices (CCDs) or photomultipliers and the like. Similarly, enzymatic labels may be detected by providing the appropriate substrates for the enzyme and detecting the resulting reaction product. Colorimetric or chemiluminescent labels may be detected simply by observing the color associated with the label. Other labeling and detection systems suitable for use in the methods of the present invention will be readily apparent to those of skill in the art. Such labeled modulators and ligands can be used in the diagnosis of a disease or health condition.

The method optionally includes at least one or more washing steps, wherein the bound TGF-β composition is washed prior to measuring protein binding to reduce background measurements caused by unbound polypeptides. Washing of the TGF-β after incubation of the polypeptide composition and before detection of TGF-β is performed in appropriate buffer plus detergent. Suitable detergents include, but are not limited to alkyldimethylamine oxides, alkyl glucosides, alkyl maltosides, alkyl sulfates (such as sodium dodecyl sulfate (SDS)), NP-40, alkyl thioglucosides, betaines, bile acids, CHAP series, digitonin, glucamides, lecithins/lysolecithins, nonionic polyoxyethylene-based detergents, including TRITON-X, polysorbates, such as TWEEN® 20 and TWEEN® 80, BRIJ®, GENAPOL® and THESIT®, quaternary ammonium compounds, and the like. See also Current Protocols in Protein Science, Appendix 1B, Suppl. 11, 1998, John Wiley and Sons, Hoboken, N.J. Suitable detergents can be determined using routine experimentation (see Neugebauer, J., A Guide to the Properties and Use of Detergents in Biology and Biochemistry, Calbiochem-Novabiochem Corp., La Jolla, Calif., 1988).

### Methods of Administering the Fibrin Sealant

It is contemplated that the fibrin sealant useful in the invention is administered to a subject using techniques well-known in the art, for example by injection or spray at the desired site, endoscopically, using a sponge-like carrier, pre-formed sealant or other methods known in the art. In one embodiment, the sealant is injected or sprayed and allowed to form a gel in situ.

These fibrin sealants are contemplated for administration to subjects who would benefit from the sustained/controlled release of TGF-β in vivo, as would be apparent to one of ordinary skill in the art, including but not limited to the conditions set out below. In one embodiment, the patient is suffering from a musculoskeletal disease, including, but not limited to, diseases of the muscles, associated ligaments, other connective tissue, and of the bones and cartilage; a soft tissue disease or disorder, including but not limited to disorders affecting, muscles, fibrous tissues, fat, blood vessels, and synovial tissues; or a cardiovascular disease.

In one embodiment, the fibrin sealants serve as a replacement for bone grafts, and thus may be applied in many of the same indications, including, but not limited to, spinal fusion cages, healing of non-union defects, bone augmentation, bone fracture repair acceleration, bone tissue reconstruction, and dental regeneration. Additionally, in another embodiment, the sealants can be used in implant integration. In implant integration, implants can be coated with a fibrin sealant inducing the neighboring bone area to grow into the surface of the implant and preventing loosening and other associated problems. In another embodiment, growth factor-enriched matrices can be used for healing chronic wounds in skin.

Additional bone or cartilage disorders or conditions include, but are not limited to, osteoarthritis, osteoporosis, osteodystrophy, rickets, osteomalacia, McCune-Albright syndrome, Albers-Schonberg disease, Paget's disease, rheumatoid arthritis, osteoarthritis, cartilage damage, periprosthetic osteolysis, osteogenesis imperfecta, metastatic bone disease, osteochondroma, osteogenesis, osteomyelitis, osteopathy, osteopetrosis, osteosclerosis, polychondritis, articular cartilage injuries, chondrocalcinosis, chondrodysplasias, chondromalacia patella, chondrosarcoma, costochondritis, enchondroma, hallux rigidus, meniscus injuries, hip labral tear, osteochondritis dissecans (ocd), relapsing polychondritis or any condition that benefits from stimulation of bone or cartilage formation.

Additional connective tissue disorders include, but are not limited to, Ehlers-Danlos syndrome, Marfan syndrome, scleroderma, cutis laxa, Dupuytren's disease, limited scleroderma, mixed connective tissue disease, Stickler syndrome and other connective tissue disease.

The fibrin sealant comprising a TGF protein is also useful to treat soft tissue diseases or conditions, including but not limited to tendonitis, bursitis, myofascial syndrome, rheumatic diseases affecting soft tissue, Tietze's syndrome, costochondritis, fasciitis, enthesitis, structural disorders, sarcoma and other conditions affecting soft tissue.

The fibrin sealant comprising a TGF protein is also useful to treat cardiovascular diseases or conditions, including but not limited to, ischemia/reperfusion, myocardial infarction, congestive heart failure, atherosclerosis, hypertension, restenosis, arterial inflammation, coronary artery disease (CAD), stroke, vessel or heart calcification, thrombosis, peripheral vascular disease, vascular wall remodeling, ventricular remodeling, rapid ventricular pacing, coronary microembolism, tachycardia, bradycardia, pressure overload, aortic bending, coronary artery ligation, vascular heart disease, valvular disease, including but not limited to, valvular degeneration caused by calcification, rheumatic heart disease, endocarditis, or complications of artificial valves; atrial fibrillation, long-QT syndrome, sinus node dysfunction, angina, heart failure, hypertension, atrial fibrillation, atrial flutter, pericardial disease, including but not limited to, pericardial effusion and pericarditis; cardiomyopathies cardiac hypertrophy or cardiovascular developmental disorders.

### Kits

Kits are also contemplated within the scope of the invention. A typical kit can comprise a fibrin sealant comprising an FC and a thrombin component. In one embodiment the kit further comprises a TGF-β protein for incorporation into the fibrin sealant. In one aspect, each component may be included in its own separate storage container, vial or vessel. In a related aspect, the TGF-β may be in admixture with the FC component and the thrombin component may be in a separate storage container. In a related embodiment, the storage container is a vial, a bottle, a bag, a reservoir, tube, blister, pouch, patch or the like. One or more of the constituents of the formulation may be lyophilized, freeze-dried, spray freeze-dried, or in any other reconstitutable form. Various reconstitution media can further be provided if desired.

The components of the kit may be in either frozen, liquid or lyophilized form. It is further contemplated that the kit contains suitable devices for administering the fibrin gel to a subject. In a further embodiment, the kit also contains instructions for preparing and administering the fibrin sealant.

Additional aspects and details of the invention will be apparent from the following examples, which are intended to be illustrative rather than limiting.

### EXAMPLES

### Example 1

### Materials and Methods

Eight different formulations of fibrin gels (TISSEEL VH™ or VH S/D™) (S/D being an added virus inactivation step to provide added safety), Baxter AG, Vienna, Austria) were prepared using different concentrations of FC and Thrombin, from 5-40 mg/ml and 2-250 IU/ml, respectively (final concentrations in the gels). Fibrin gels (0.3 ml total) were prepared in 24-well culture plates. The FC component contained aprotinin, a fibrinolysis inhibitor, at 3000 KIU/ml.

Recombinant human (rh) TGF-β1 (R&D Systems) at 5 to 40 ng/0.3 ml gel, or rhTGF-β2 (R&D Systems, Minneapolis, Minn.) at 15 ng/0.3 ml gel, or rhTGF-β3 (R&D Systems) at 15 ng/0.3 ml gel, was added in the FC component at the time of the gel preparation. All gels were incubated at 37° C. in 5% CO₂ for up to 10 days with standard HMSC growth medium (Lonza Walkersville Inc, formerly Cambrex Bio Science Walkersville Inc., Walkersville, Md.). Medium was changed every day. Medium samples were frozen until tested for the amount of TGF-β by enzyme linked immunosorbant assay (ELISA) (R&D Systems). In order to verify full recovery of the initially added TGF-β1, some gels were dissolved using urokinase after the 10 days incubation and supernatants were also tested by ELISA.

Different release kinetics were conducted:

1. The effects of TGF-β1 amount on the release kinetic were analyzed using a single fibrin gel formulation (FC concentration of 25 mg/ml and Thrombin concentration of 2 IU/ml, final concentrations in the gel) made of TISSEEL VH™.

2. The effects of FC concentration with a fixed Thrombin concentration (2 IU/ml, final concentration in the gels) on TGF-β1 release kinetic were analyzed using four different FC concentrations of TISSEEL VH™.

3. The effects of FC concentration with a fixed Thrombin concentration (2 IU/ml, final concentration in the gels) on TGF-β1 release kinetic were analyzed using four different FC concentrations of TISSEEL VH S/D™.

4. The effects of Thrombin concentration with a fixed FC concentration (25 mg/ml, final concentration in the gels) on TGF-β1 release kinetic were analyzed using four different Thrombin concentrations of TISSEEL VH™.

5. The effects of varying the lot numbers of TISSEEL VH™ or TISSEEL VH S/D™ on the release kinetic of TGF-β1 were analyzed using a single fibrin gel formulation (FC concentration of 20 mg/ml and Thrombin concentration of 2 IU/ml, final concentrations in the gels).

6. The effects of FC concentration with a fixed Thrombin concentration (2 IU/ml, final concentration in the gels) on TGF-β2 release kinetics were analyzed using four different FC concentrations of TISSEEL VH™.

7. The effects of FC concentration with a fixed Thrombin concentration (2 IU/ml, final concentration in the gels) on TGF-β3 release kinetics were analyzed using four different FC concentrations of TISSEEL VH™.

The effects of TGF-β1 (15 ng/0.3 ml gel) added in fibrin gels on HMSC seeded on the gel surface were analyzed by fluorescence microscopy. Gels without TGF-β1 added in FC were used as controls. About 10,000 HMSC (Lonza Walkersville Inc.) were seeded on top of gels prepared in 24 well-plates, with a FC concentration from 5-40 mg/ml and a Thrombin concentration from 2-250 IU/ml (final concentrations in the gels). Gels with cells were stained with calcein/ethidium bromide dye at days 1, 4, 7 and 10 in order to observe the morphology and migration of the cells in presence or absence of added TGF-β1 in the gels.

In order to test the biological activity of the released TGF-β1, medium supernatants from gels at day 3 (initially containing no added TGF-β1 (control) or an added 15 ng of TGF-β1/0.3 ml gel, prepared with 20 mg/ml of FC and 2 IU/ml of Thrombin (final concentrations in the gels)) were used as culture medium for HMSC monolayers. Cells cultured in medium containing freshly added TGF-β1 served as positive control. Changes in cell morphology up to 7 days were analyzed by light and fluorescence microscopy after staining with calcein dye. Cell proliferation was also analyzed after staining with calcein dye by measuring the overall fluorescence intensity (measured by optical density) of the cell monolayers. Results were normalized on day 1. Cell differentiation was analyzed by Alcian Blue staining, used to show the presence of glycoaminoglycans (for chondrogenesis), and alkaline phosphatase (ALP) activity as well as Alizarin Red staining (for osteogenesis). ALP activity results (first calculated in IU/ml) were normalized on proliferation. Some of the gels had been prepared with no added TGF-β1 for control samples in order to insure that any changes observed with medium supernatants from gels with added TGF-β1 were indeed induced by the released TGF-β1 and not by some other bioactive components that could be released from the gels themselves.

Finally, in order to analyze the effects of TGF-β1 on the behavior of HMSC seeded into fibrin gels and Human Umbilical Vascular Endothelial Cells (HUVEC, Lonza Walkersville Inc.) seeded on the gel surface, gels containing 10 mg/ml of FC and 2 IU/ml of Thrombin (final concentrations in the gels) were prepared with single culture cells (HMSC or HUVEC) or co-culture cells at a HMSC:HUVEC ratio of 4:1. Recombinant human TGF-β1 (5 ng/0.3 ml gel, R&D Systems) was added in the FC in half of the co-culture gels at the time of gel preparation. Gels were incubated at 37° C. in 5% CO₂ for up to 21 days using a standard endothelial cell growth culture medium (Lonza Walkersville Inc.) with a serum supplement. Analysis of cell morphology and proliferation, as well as osteogenic differentiation, was performed at days 1, 7, 14 and 21. Cell morphology including the reorganization of HUVEC into interconnected cell-cell networks (early events of angiogenic differentiation) was observed by fluorescence microscopy after staining with Calcein dye. Cell proliferation was measured by the fluorescence intensity of the cell suspensions after dissolving the fibrin gels in a purified, concentrated bovine trypsin solution. Finally, ALP activity was measured as an early marker of osteogenic differentiation. Statistical analysis was performed using the ANOVA test with 5% as the level of significance.

### Example 2

### Release Kinetics of TGF-β1 Added at Different Amounts in Fibrin Gels

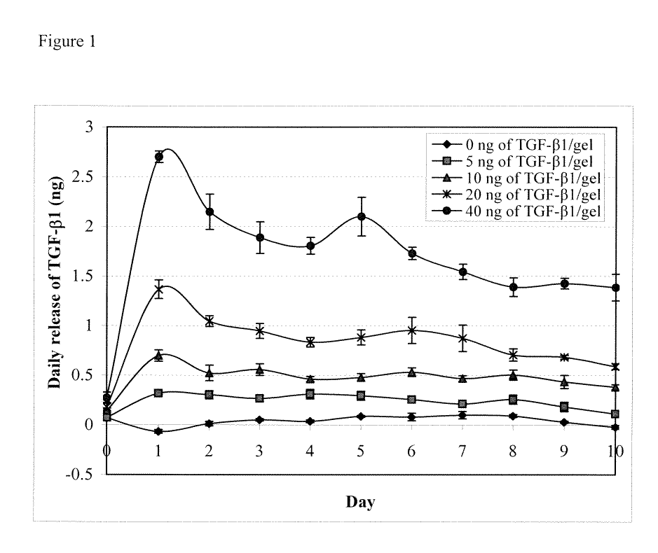

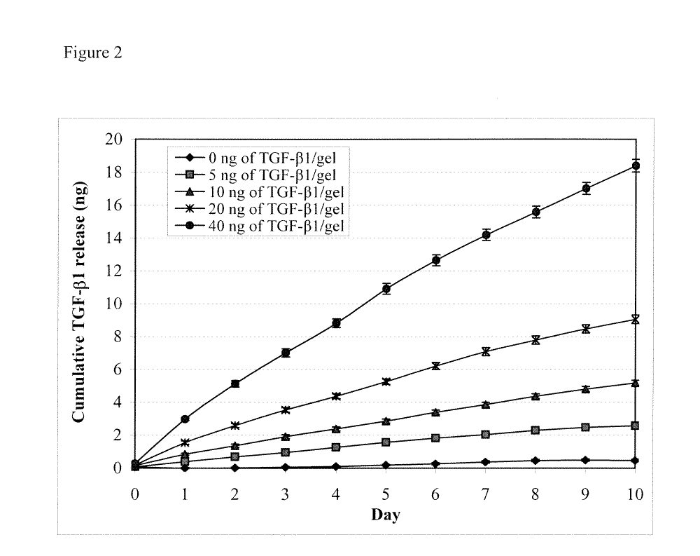

The effects of TGF-β1 amount on its release kinetics from fibrin gels were analyzed using a single fibrin gel formulation (FC concentration of 25 mg/ml and Thrombin concentration of 2 IU/ml, final concentration in the gels) made of TISSEEL VH™. Analysis of the effects of TGF-β1 amount on the daily (FIG. 1) and cumulative (FIG. 2) TGF-β1 release from the gels showed that the level of TGF-β1 release was proportional to the amount of growth factor initially added to the FC component. Overall, results with this fibrin gel formulation showed that only about 45% to 52% of the initially added TGF-β1 was released after 10 days (FIG. 2). In other words, about 48% to 55% of the initially added TGF-β1 was retained in the fibrin gels after 10 days. If considering the cumulative release of TGF-β1 after only 3 days, the minimum retention was about 80%.

### Example 3

### Release Kinetics of TGF-β1 from Fibrin Gels with Varying Concentrations of FC or Thrombin

Effects of FC concentration with a fixed Thrombin concentration (2 IU/ml) (TISSEEL VH) on TGF-β1 release kinetics: To determine the effects of FC concentration on the TGF-β1 release kinetics when using TISSEEL VH™ Fibrin Sealant, release was analyzed using gels having a fixed Thrombin concentration (2 IU/ml) over four different FC concentrations (5, 10, 20 and 40 mg/ml, final concentrations in the gels) of TISSEEL VH.

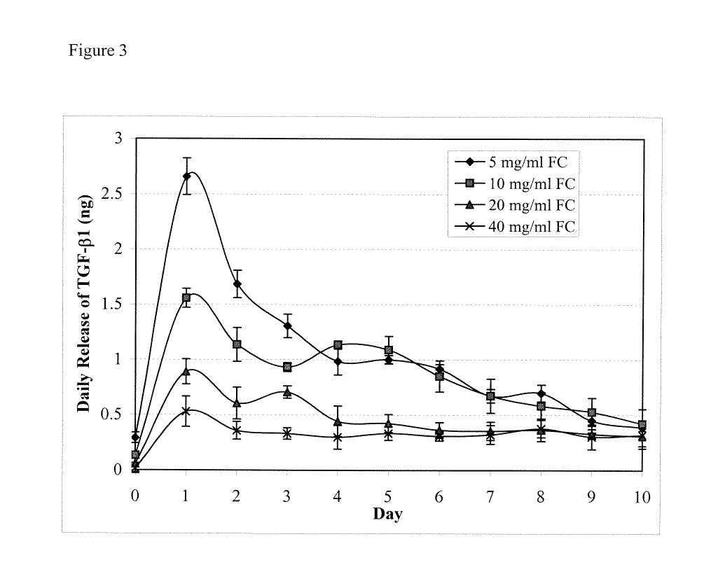

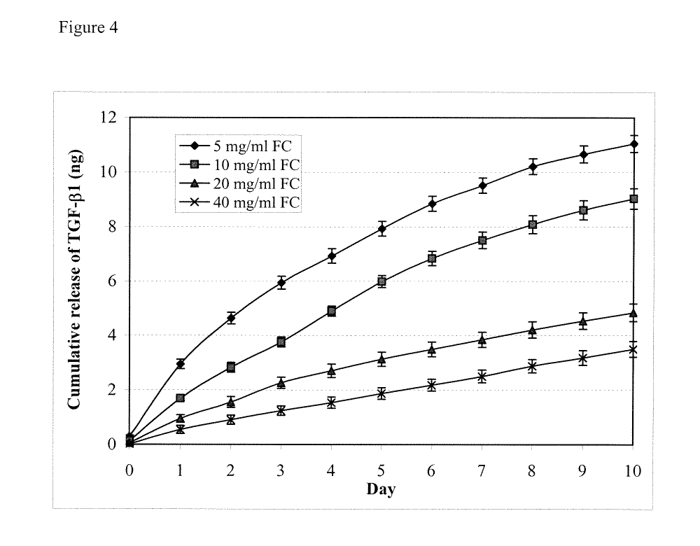

Analysis of the daily TGF-β1 release by ELISA showed a spike release at day 1 and a decrease in the release up to day 10 for all FC concentrations analyzed (FIG. 3). The release was significantly higher with gels containing lower FC concentrations, indicating an effect of FC concentration on the release kinetic, and a potential binding affinity of TGF-β1 with the FC component proteins of the fibrin gels. The cumulative release of TGF-β1 by day 10 was also lower than the initial added amount of the growth factor (15 ng), with a maximum percentage of cumulative release of about 75% (with 5 mg/ml of FC), and a minimum of about 25% (with 40 mg/ml of FC) (FIG. 4). Therefore, these results showed a minimum retention of about 25% (with 5 mg/ml of FC) and a maximum retention of about 75% (with 40 mg/ml of FC) after 10 days. If considering the cumulative release of TGF-β1 after only 3 days, the minimum retention was about 60% (with 5 mg/ml of FC) and the maximum retention about 90% (with 40 mg/ml of FC).

Effects of FC concentration with a fixed Thrombin concentration (2 IU/ml)) (TISSEEL VH S/D™) on TGF-β1 release kinetics: To determine the effects of FC concentration on the TGF-β1 release kinetics when using TISSEEL VH S/D™ Fibrin Sealant, release was analyzed using gels having a fixed Thrombin concentration (2 IU/ml) over four different FC concentrations (5, 10, 20 and 40 mg/ml, final concentrations in the gels) of TISSEEL VH S/D™.

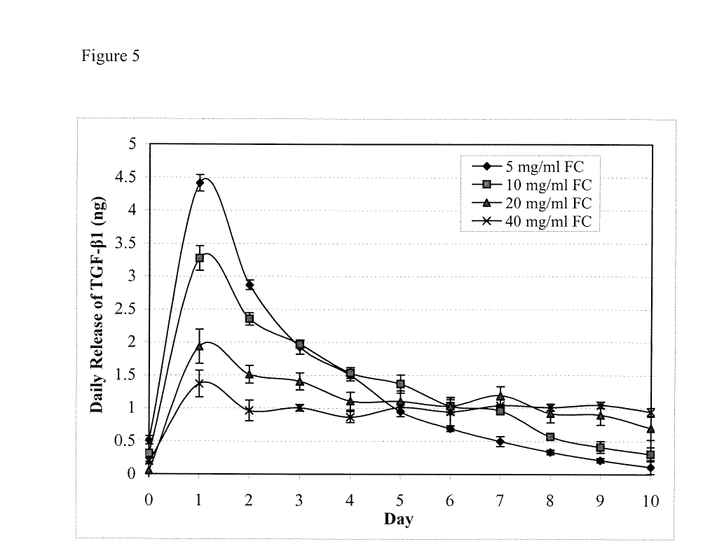

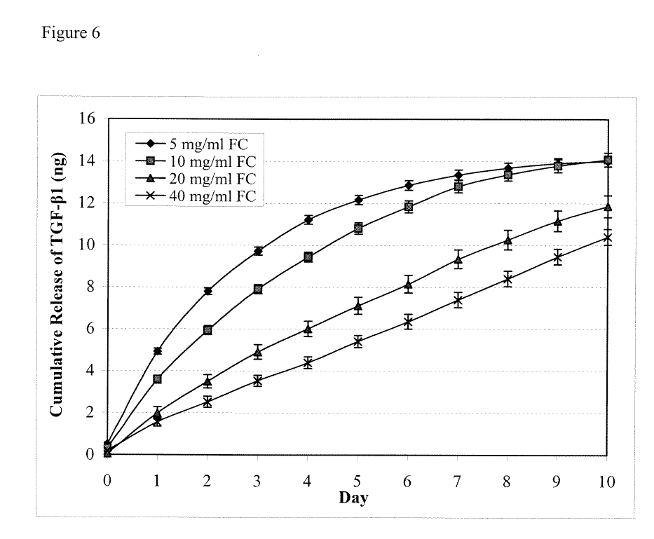

Analysis of the daily TGF-β1 release by ELISA showed a spike release at day 1 and a decrease in the release up to day 10 for all FC concentrations analyzed (FIG. 5). The release was significantly higher with gels containing lower FC concentrations, indicating an effect of FC concentration on the release kinetics, and a potential binding affinity of TGF-β1 with the FC component proteins of the fibrin gels. The cumulative release of TGF-β1 by day 10 reached about 70% (with 40 mg/ml of FC) to almost 100% (more than 93%) with the two lowest FC concentrations (5 mg/ml and 10 mg/ml, final concentrations in the gels) (FIG. 6). Therefore, the maximum retention was about 30% (with 40 mg/ml of FC) after 10 days. If considering the cumulative release of TGF-β1 after only 3 days, the minimum retention was about 35% (with 5 mg/ml of FC) and the maximum retention about 75% (with 40 mg/ml of FC).

Effects of Thrombin concentration with a fixed FC concentration (25 mg/ml) on TGF-β1 release (TISSEEL VH™): To determine the effects of Thrombin concentration on the TGF-β1 release kinetics, release was analyzed using gels having a fixed FC concentration (25 mg/ml) over four different Thrombin concentrations (2, 10, 50, 250 IU/ml, final concentrations in the gels) of TISSEEL VH.

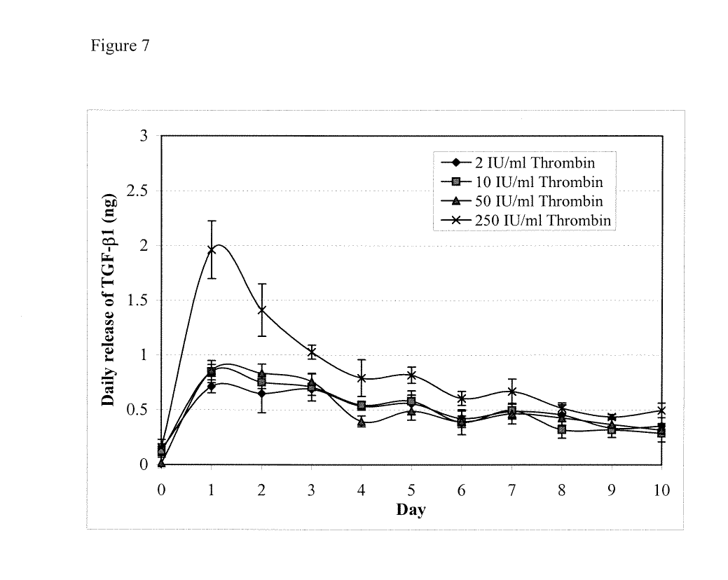

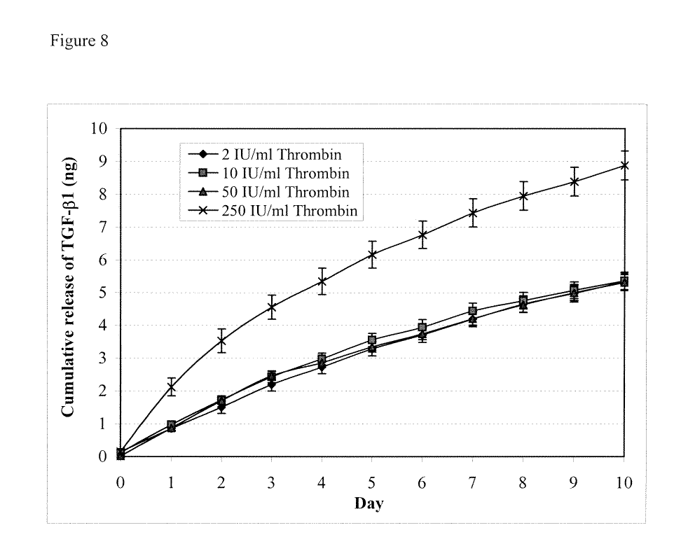

Analysis of the daily TGF-β1 release by ELISA showed a spike release at day 1 and a decrease in the release up to day 10 for all Thrombin concentrations analyzed (FIG. 7). The release was similar with 2, 10 and 50 IU/ml of Thrombin (about 15% release (i.e. 85% retention) after 3 days, and about 35% release (i.e. 65% retention) after 10 days), suggesting that thrombin concentration has a lesser effect than FC concentration on TGF-β1 release. TGF-β1 release was only significantly higher with the highest thrombin concentration (250 IU/ml) most likely because of a more heterogeneous structure of the gels with such a high thrombin concentration. Similar to the effects of FC, the cumulative release of TGF-β1 by day 10 was lower than the initial added amount of the growth factor (FIG. 8).

Effects of varying the lot numbers of TISSEEL VH™ or TISSEEL VH S/D™ on TGF-β1 release: The effects of varying the lot numbers of TISSEEL VH™ or TISSEEL VH S/D™ on the release kinetics of TGF-β1 were analyzed using a single fibrin gel formulation (FC concentration of 20 mg/ml and Thrombin concentration of 2 IU/ml, final concentrations in the gels).

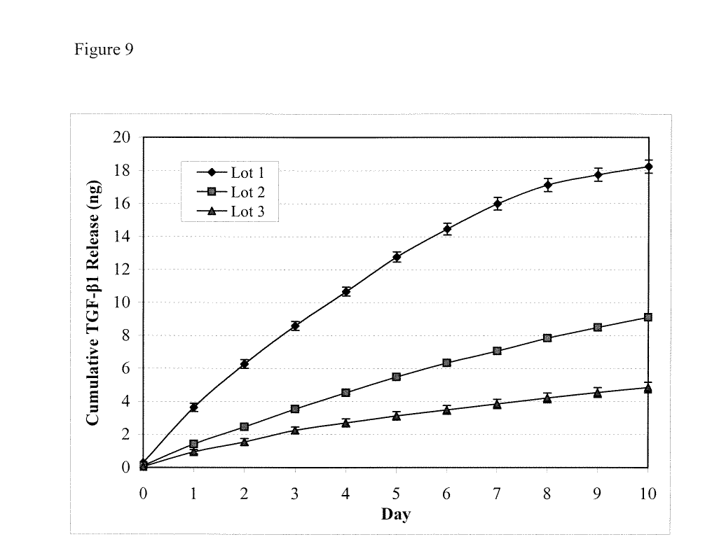

Analysis of the effects of varying FC lot number from TISSEEL VH™ showed a cumulative release from about 32% to full release depending on the lot number, after 10 days (FIG. 9). In other words, results showed negligible retention to a maximum retention of 68% depending on the lot number, after 10 days. These results corroborated with Factor XIII content in these different lots, the lowest % release (i.e. highest retention) being obtained with the lot number containing the highest amount of Factor XIII (lot 1 contained <1 U/ml of Factor XIII, lot 2 contained 33.9 U/ml and lot 3 contained 42.2 U/ml). If considering the cumulative release of TGF-β1 after only 3 days, the minimum retention was about 43% (with lot 1) and the maximum retention about 85% (with lot 3).

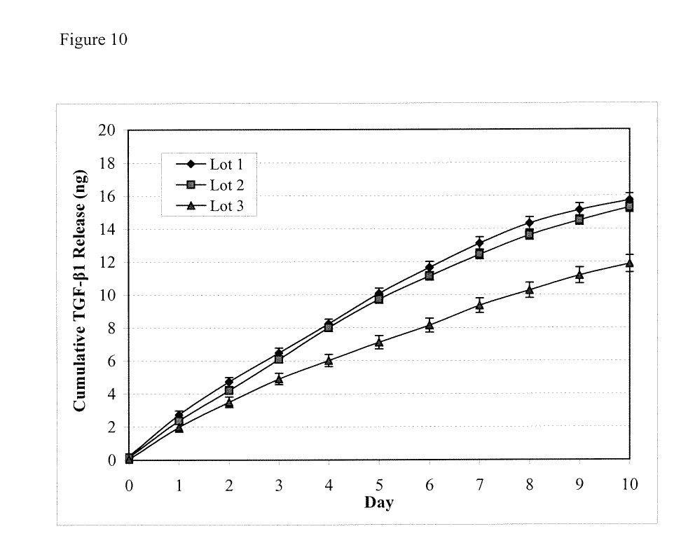

Analysis of the effects of varying FC lot number from TISSEEL VH S/D™ showed a cumulative release from about 80% to full release depending on the lot number, after 10 days (FIG. 10). Therefore, results showed a negligible retention to a maximum retention of 20% depending on the lot number, after 10 days. Factor XIII content in these lots was lower than 3 U/ml. If considering the cumulative release of TGF-β1 after only 3 days, the minimum retention was about 57% (with lot 1) and the maximum retention about 67% (with lot 3).

### Example 4

### Release Kinetics of Other Isoforms of TGF-1 (TGF-β2 and TGF-β3)

Release kinetics of TGF-β2: The effects of FC concentration on the TGF-β2 release kinetics from TISSEEL VH™ Fibrin Sealant were analyzed using gels having a fixed Thrombin concentration (2 IU/ml) over four different FC concentrations (5, 10, 20 and 40 mg/ml, final concentrations in the gels) of TISSEEL VH™.

ELISA results showed that the cumulative TGF-β2 release was significantly higher with gels containing lower FC concentrations, at least at early time-points, indicating an effect of FC concentration on TGF-β2 release kinetics, and a potential binding affinity of TGF-β2 with the FC component proteins of the fibrin gels. By day 10, the release reached 85% (with 40 mg/ml of FC) to 100% with the other formulations containing lower FC concentrations. In other words, these results showed a maximum retention of about 15% (with 40 mg/ml of FC) after 10 days. If considering the cumulative release of TGF-β2 after only 3 days, the minimum retention was about 25% (with 5 mg/ml of FC) and the maximum retention about 70% (with 40 mg/ml of FC).

Release kinetics of TGF-β3: The effects of FC concentration on the TGF-β3 release kinetics from TISSEEL VH™ Fibrin Sealant were analyzed using gels having a fixed Thrombin concentration (2 IU/ml) over four different FC concentrations (5, 10, 20 and 40 mg/ml, final concentrations in the gels) of TISSEEL VH™.

ELISA results showed that the cumulative TGF-β3 release was significantly higher with gels containing lower FC concentrations, indicating an effect of FC concentration on TGF-β3 release kinetics, and a potential binding affinity of TGF-β3 with the FC component proteins of the fibrin gels. The cumulative release of TGF-β3 by day 10 was also lower than the initial added amount of the growth factor, with a maximum percentage of cumulative release of about 75% (with 5 mg/ml of FC), and a minimum of about 30% (with 40 mg/ml of FC). In other words, these results showed a minimum retention of about 25% (with 5 mg/ml of FC) and a maximum retention of about 70% (with 40 mg/ml of FC) after 10 days. If considering the cumulative release of TGF-β3 after only 3 days, the minimum retention was about 55% (with 5 mg/ml of FC) and the maximum retention about 90% (with 40 mg/ml of FC).

### Example 5

### Effects of TGF-β1 on HMSC Seeded on the Surface of Fibrin Gels

Human mesenchymal stem cells (HMSC) are pluripotent progenitor cells that can differentiate into different specialized tissue cell types including chondrocytes, osteoblasts, adipocytes and myocytes (Caplan A I, J Orthop Res 9: 641-650, 1991). The commitment and differentiation of these cells are modulated by a variety of factors, including cell interactions but also specific growth factors. Members of TGF-β family of growth factors have been identified as regulators of MSC maturation. In particular, TGF-β1 is involved in cartilage and bone development, most likely by inducing the differentiation of MSC into the chondrogenic or osteogenic lineage (Centrella et al., Endocrine Rev, 15: 27-39, 1994). In addition, TGF-β1 is an important angiogenic factor involved in the different aspects of angiogenesis, a critical process during bone growth. Many studies have been reported on the importance of the TGF-β signaling pathway in angiogenesis and vascular remodeling (Bertolino et al., Chest 128: 6, 2005). Finally, TGF-β has been shown to play an important role in capillary morphogenesis and the maintenance of vessel wall integrity (Pepper M S. Cytokine & Growth Factor Reviews 8(1): 21-43, 1997).

The effects of TGF-β1 (15 ng/0.3 ml gel) added in fibrin gels on HMSC seeded on the gel surface were analyzed by fluorescence microscopy. Gels without TGF-β1 added in FC were used as controls. About 10,000 HMSC (Lonza Walkersville Inc.) were seeded on top of gels prepared in 24 well-plates, with a FC concentration from 5-40 mg/ml and a Thrombin concentration from 2-250 IU/ml (final concentrations in the gels). Gels with cells were stained with calcein/ethidium bromide dye at days 1, 4, 7 and 10 in order to observe the morphology and migration of the cells in presence or absence of added TGF-β1 in the gels.

Fluorescence microscopy analysis of the gels with HMSC seeded on the gel surface showed that HMSC proliferated well and formed a monolayer as early as day 7, for all the gel formulations analyzed, with or without the addition of TGF-β1 in the gels. Results also showed that added TGF-β1 in the fibrin gels influenced HMSC morphology and migration into the gels when cultured on the gel surface. Indeed, cells displayed an elongated shape on gels prepared with no added TGF-β1, but had a more squared to polygonal shape when seeded on the surface of the gels prepared with added TGF-β1. The more squared to polygonal shape of the MSC in the presence of TGF-β1 added in the gels indicates their differentiation into the osteogenic and/or chondrogenic phenotype. Also, some cells migrated inside the gels prepared with no added TGF-β1 whereas they remained mostly on the surface of the gels prepared with added TGF-β1.

The absence of cell migration inside the gels prepared with added TGF-β1 indicates the ability of the fibrin gels to deliver TGF-β1 to the cells seeded onto the gels. By means of a fibrin gel of the invention, TGF-β1 can be delivered to cells in the surrounding area in an in vivo setting.

### Example 6

### Biological Activity of Released TGF-β1 on HMSC Monolayers In Vitro

In order to test the biological activity of the released TGF-β1, medium supernatants from gels at day 3 (initially containing no added TGF-β1 (control) or an added 15 ng of TGF-β1/0.3 ml gel prepared with 20 mg/ml of FC and 2 IU/ml of Thrombin, final concentrations in the gels) were used as culture medium for HMSC monolayers. Cells cultured in medium containing freshly added TGF-β1 served as positive control. Changes in cell morphology up to 7 days were analyzed by light and fluorescence microscopy after staining with calcein dye. Cell proliferation was also analyzed after staining with calcein dye by measuring the overall fluorescence intensity (measured by optical density) of the cell monolayers. Results were normalized on day 1. Cell differentiation was analyzed by Alcian Blue staining, used to show the presence of glycoaminoglycans (for chondrogenesis), and Alkaline Phosphatase (ALP) activity as well as Alizarin Red staining (for osteogenesis). ALP activity results (first calculated in IU/ml) were normalized on proliferation. Some of the gels had been prepared with no added TGF-β1 for control samples in order to insure that any changes observed with medium supernatants from gels with added TGF-β1 were indeed induced by the released TGF-β1 and not by some other bioactive components that could be released from the gels themselves.

HMSC monolayers cultured in medium supernatants from gels with added TGF-β1 (i.e. in medium containing released TGF-β1) at day 3 showed a change in morphology as early as day 4 and even more pronounced at day 7. They had a more squared to polygonal shape than those cultured in medium supernatants from gels with no added TGF-β1, and a similar shape to those cultured in medium containing freshly added TGF-β1 (positive control).

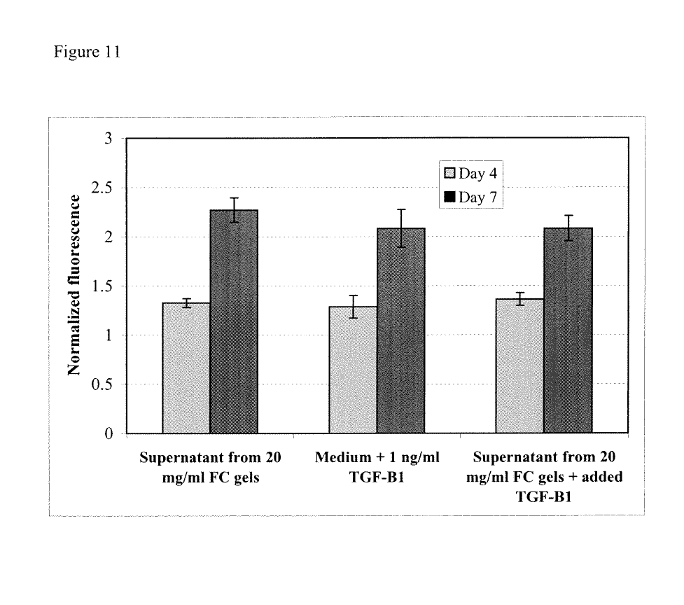

Cell proliferation at days 4 and 7 were normalized on proliferation at day 1 (baseline). The proliferation of the cells cultured in medium supernatants from gels with added TGF-β1 (i.e. in medium containing released TGF-β1) tended to be lower than that of the cells cultured in medium supernatants from gels with no added TGF-β1, and similar to the one of the cells cultured in medium with freshly added TGF-β1 (FIG. 11).

The change of HMSC morphology into a more squared to polygonal shape after culture in monolayers in medium supernatants from gels with added TGF-β1 (i.e., in medium containing released TGF-β1) indicates cell differentiation, in parallel to a tendency to exhibit lower proliferation compared to cells cultured in medium supernatants from gels with no added TGF-β1.

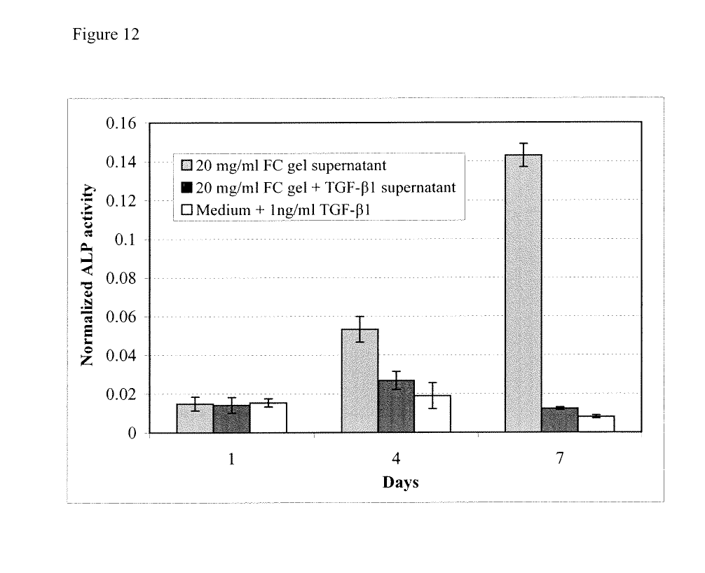

Cell differentiation into the chondrogenic or osteogenic phenotypes was accessed by staining with Alcian blue (for chondrogenesis) and ALP activity as well as Alizarin Red (for osteogenesis). Alcian blue staining increased with time in HMSC cultured in medium supernatants from gels with added TGF-β1 (i.e. in medium containing released TGF-β1), and by day 7, the amount of staining was significantly higher than that in HMSC cultured in medium supernatants from gels with no added TGF-β1 and similar to the one in HMSC cultured in medium with freshly added TGF-β1. Alizarin Red staining remained negative in all samples. ALP activity decreased between days 4 and 7 in the HMSC cultured in medium supernatants from gels with added TGF-β1 (FIG. 12). It was significantly lower than the level detected in HMSC cultured in medium supernatants from gels with no added TGF-β1 as early as day 4 (p=0.005 at day 4 and p=3E-06 at day 7), and at similar level to the one in HMSC cultured in medium with freshly added TGF-β1.

Changes in cell morphology, proliferation and chondrogenic differentiation demonstrate that the TGF-β1 is still biologically active after its release from the gels.

### Example 7

### Effects of TGF-β1 on HMSC Seeded Inside Fibrin Gels and HUVEC Seeded on the Surface of Fibrin Gels

In order to analyze the effects of TGF-β1 on the behavior of HMSC seeded into fibrin gels and Human Umbilical Vascular Endothelial Cells (HUVEC, Lonza Walkersville Inc.) seeded on the gel surface, gels containing 10 mg/ml of FC and 2 IU/ml of Thrombin (final concentrations in the gels) were prepared with single culture cells (HMSC or HUVEC) or co-culture cells at a HMSC:HUVEC ratio of 4:1. Recombinant TGF-β1 (5 ng/0.3 ml gel) was added in the FC in half of the co-culture gels at the time of gel preparation. Gels were incubated at 37° C. in 5% CO₂ for up to 21 days using a standard endothelial cell growth culture medium (Lonza Walkersville Inc,) with a serum supplement. Analysis of cell morphology and proliferation, as well as osteogenic differentiation, was performed at days 1, 7, 14 and 21. Cell morphology including the reorganization of HUVEC into interconnected cell-cell networks (early events of angiogenic differentiation) was observed by fluorescence microscopy after staining with Calcein dye. Cell proliferation was measured by the fluorescence intensity of the cell suspensions after dissolving the fibrin gels in a purified, concentrated bovine trypsin solution. Finally, a standard alkaline phosphatase (ALP) assay was performed as an early marker of osteogenic differentiation.

Fluorescence microscopy analysis showed that HMSC were more evenly dispersed and had a more elongated shape when seeded in single culture gels or in co-culture gels with added TGF-β1. HSMC were smaller and tended to migrate towards the bottom of the co-culture gels without added TGF-β1. HUVEC reorganization into interconnected cell-cell networks (early events of angiogenic differentiation) started earlier and happened to a larger extend in single culture gels and co-cultured gels containing TGF-β1 compared to co-culture gels without added TGF-β1.

Cell proliferation increased with time. No significant difference was observed between gels with and without added TGF-β1. ALP activity for all the HMSC-containing conditions increased with time. Its level tended to be higher in gels with added TGF-β1 compared to that in gels with no added TGF-β1, except at day 7. This indicated an increase in early osteogenic differentiation after 14 days in gels with added TGF-β1, but it could also reflect a possible higher number of HMSC in presence of TGF-β1. Because the performed proliferation assay did not differentiate between the two cell types, ALP activity level could not be directly related to the overall number of HMSC.

Numerous modifications and variations of the invention as set forth in the above illustrative examples are expected to occur to those skilled in the art. Consequently only such limitations as appear in the appended claims should be placed on the invention.

1. A method for modifying M release of a transforming growth factor-beta (TGF-β) protein, said protein selected from the group consisting of TGF-β1, TGF-β2 and TGF-β3, from a fibrin sealant, wherein the fibrin sealant is produced by admixture of a fibrinogen complex component, a thrombin component and a TGF-β component, the method comprising,
a) determining an amount of TGF-β released from a first fibrin sealant having a known initial amount of TGF-β and a known final concentration of fibrinogen complex, and
b) modifying the known final concentration of fibrinogen complex used in the first fibrin sealant of step (a) to produce a second fibrin sealant,
wherein increasing the concentration of the fibrinogen complex in the second sealant compared to the known final concentration of fibrinogen complex in the first sealant decreases the rate of TGF-β release from the second sealant as compared to the release of TGF-β from the first sealant of step (a), and wherein the second sealant has the same initial amount of TGF-β as the first sealant in step (a), or
wherein decreasing the concentration of fibrinogen complex in the second sealant compared to the known final concentration of fibrinogen complex in the first sealant increases the rate of TGF-β release from the second sealant as compared to the release of TGF-β from the first sealant of step (a), and wherein the second sealant has the same initial amount of TGF-β as the first sealant in step (a).

2. (canceled)

3. The method of claim 1 wherein the final fibrinogen complex concentration in the first or second sealant is within the range of about 1 mg/ml to about 150 mg/ml.

4. The method of claim 3 wherein the final fibrinogen complex concentration in the first or second sealant is within the range of about 5 mg/ml to about 75 mg/ml.

5. The method of claim 1 wherein the final fibrinogen complex concentration in the first fibrin sealant differs from the final fibrinogen complex concentration in the second sealant by about 1 mg/ml to about 149 mg/ml.

6. The method of claim 1 or wherein the final fibrinogen complex concentration in the first fibrin sealant differs from the fibrinogen complex concentration in the second sealant by about 5 mg/ml to about 75 mg/ml.

7. The method of claim 1 or wherein the final fibrinogen complex concentration in the first fibrin sealant differs from the fibrinogen complex concentration in the second sealant by about 10 mg/ml to about 60 mg/ml.

8. The method of claim 1 wherein the final concentration of the thrombin component in the first or second sealant is within the range of about 1 IU/ml to 250 IU/ml.

9. The method of claim 1 wherein the final concentration of TGF-β3 is in the range of about 1 ng/ml to about 1 mg/ml.

10. A method for controlled release of a transforming growth factor-beta (TGF-β3) protein, said protein selected from the group consisting of TGF-β1, TGF-β2 and TGF-β3, in a patient in need thereof, comprising administering to said patient a fibrin sealant comprising TGF-β,
wherein at least 25% of the TGF-β is retained in the fibrin sealant for at least 3 days, or wherein at least 20% of the TGF-β is retained in the fibrin sealant for at least 10 days.

11. The method of claim 10 wherein the fibrin sealant is produced by combining a fibrinogen complex (FC) component and a thrombin component in admixture.

12. The method of claim 11 wherein the TGF-β is added to the FC component before admixture of the FC component with the thrombin component.

13. The method of claim 10 wherein at least 35% to 90% of the TGF-β is retained for at least 3 days.

14. The method of claim 10 wherein at least 45% to 75% of the TGF-β is retained in the fibrin sealant for at least 3 days.

15. The method of claim 10 wherein at least 60% of the TGF-β is retained in the fibrin sealant for at least 3 days.

16. The method of claim 10 wherein the TGF-β released is biologically active.

17-19. (canceled)

20. The method of claim 10 wherein at least 25% to 75% of the TGF-β is retained for at least 10 days.

21. The method of claim 10 wherein at least 45% to 55% of said TGF-β is retained in the fibrin sealant for at least 10 days.

22. (canceled)

23. The method of claim 11 wherein the final fibrinogen complex concentration in the sealant is within the range of about 1 mg/ml to about 150 mg/ml.

24. The method of claim 11 wherein the final thrombin concentration in the sealant is within the range of about 1 IU/ml to 250 IU/ml.

25. The method of claim 11 wherein the final fibrinogen complex concentration is 40 mg/ml and the final thrombin concentration is about 2 IU/ml.

26. The method of claim 10 wherein the final TGF-β concentration in the sealant is from about 1 ng/ml to about 1 mg/ml.

27. The method of claim 10 wherein the TGF-β is TGF-β1.

28. The method of claim 27 wherein at least 60% of said TGF-β1 is retained in said fibrin sealant for at least 3 days, and wherein at least 25% of said TGF-β1 is retained in said fibrin sealant for at least 10 days.

29. The method of claim 10 wherein the TGF-β is TGF-β2.

30. The method of claim 29 wherein at least 25% of said TGF-β2 is retained in said fibrin sealant for at least 3 days.

31. The method of claim 10 wherein the TGF-β is TGF-β3.

32. The method of claim 31 wherein at least 55% of said TGF-β3 is retained in said fibrin sealant for at least 3 days, and wherein at least 25% of said TGF-β3 is retained in said fibrin sealant for at least 10 days.

33. The method of claim 10 wherein the patient is suffering from a disease selected from the group consisting of a musculoskeletal disease or disorder, a soft tissue disease or disorder and a cardiovascular disease.

34-36. (canceled)

37. A method for treating a patient suffering from a disorder or disease which would benefit from in situ controlled release of a transforming growth factor-beta (TGF-β) protein, said protein selected from the group consisting of TGF-β1, TGF-β2 and TGF-β3, said method comprising administering to said patient a fibrin sealant comprising the TGF-β protein,
wherein the fibrin sealant provides a controlled release of the TGF-β wherein at least 25% of the TGF-β is retained in the fibrin sealant for at least 3 days, or wherein at least 20% of the TGF-β is retained in the fibrin sealant for at least 10 days, and said TGF-β is released at a rate effective to treat said disorder or disease.

38. The method of claim 37 wherein the fibrin sealant is produced by combining a fibrinogen complex (FC) component and a thrombin component in admixture.

39. The method of claim 38 wherein the TGF-β is added to the FC component before admixture of the FC component with the thrombin component.

40. The method of claim 37 wherein at least 35% to 90% of the TGF-β is retained in the fibrin sealant for at least 3 days.

41. The method of claim 37 wherein at least 45% to 75% of the TGF-β is retained in the fibrin sealant for at least 3 days.

42. The method of claim 37 wherein at least 60% of the TGF-β is retained in the fibrin sealant for at least 3 days.

43. The method of claim 37 wherein at least 20% of said TGF-β is retained in the fibrin sealant for at least 10 days.

44. The method of claim 37 wherein at least 25% to 75% of the TGF-β is retained for at least 10 days.

45. The method of claim 37 wherein at least 45% to 55% of said TGF-β is retained in the fibrin sealant for at least 10 days.

46. The method of claim 37 wherein the released TGF-β is biologically active.

47. The method of claim 38 wherein the final FC concentration in the sealant is within the range of about 1 mg/ml to about 150 mg/ml.

48. The method of claim 38 wherein the final thrombin concentration in the sealant is within the range of about 1 IU/ml to 250 IU/ml.

49. The method of claim 38 wherein the final fibrinogen complex concentration is about 40 mg/ml and the final thrombin concentration is about 2 IU/ml.

50. The method of claim 37 wherein the final TGF-β concentration in the sealant is from about 1 ng/ml to about 1 mg/ml.

51. The method of claim 37 wherein the TGF-β is TGF-β1.

52. The method of claim 51 wherein at least 60% of said TGF-β1 is retained in said fibrin sealant for at least 3 days, and wherein at least 25% of said TGF-β1 is retained in said fibrin sealant for at least 10 days.

53. The method of claim 37 wherein the TGF-β is TGF-β2.

54. The method of claim 53 wherein at least 25% of said TGF-β2 is retained in said fibrin sealant for at least 3 days.

55. The method of claim 37 wherein the TGF-β is TGF-β3.

56. The method of claim 55 wherein at least 55% of said TGF-β3 is retained in said fibrin sealant for at least 3 days, and wherein at least 25% of said TGF-β3 is retained in said fibrin sealant for at least 10 days.

57. The method of claim 37 wherein the patient is suffering from a disease selected from the group consisting of a musculoskeletal disease or disorder, a soft tissue disorder and a cardiovascular disease.

58. The method of claim 57 wherein the musculoskeletal disorder is a bone disease or a bone disorder.

59. The method of claim 57 wherein the musculoskeletal disorder is a cartilage disease or a cartilage disorder.

60. The method of claim 57 wherein the patient is suffering from a cardiovascular disease.

61. A kit for preparing a fibrin sealant comprising a transforming growth factor-beta (TGF-β3) protein, said protein selected from the group consisting of TGF-β1, TGF-β2 and TGF-β3, and said fibrin sealant having a desired TGF-β release rate, the kit comprising,
a) a first vial or first storage container containing a fibrinogen complex component, wherein the vial optionally comprises a TGF-β component, and b) a second vial or second storage container having a thrombin component, said kit optionally containing a third vial or third storage container having a TGF-β component when said first vial or first storage container does not include a TGF-β component, said kit further containing instructions for use thereof.
